(19) United States
(12) Patent Application Publication
(10) Pub. No.: US 2022/0167838 A1
(43) Pub. Date: Jun. 2, 2022

(54) Triple-Wafer Dual-Band Fluorescent Zoom Adapter for Endoscope

(71) Applicant: Changchun University of Science and Technology, Changchun City (CN)

(72) Inventors: Qi Li, Changchun City (CN); Yongkun Liu, Changchun City (CN); Yang Xiang, Changchun City (CN); Meng Dong, Changchun City (CN); Tingting Li, Changchun City (CN); Zhongyu Cao, Changchun City (CN)

(21) Appl. No.: 17/457,282

(22) Filed: Dec. 2, 2021

(30) Foreign Application Priority Data
Dec. 2, 2020 (CN) 202011392144.8

(51) Int. Cl. A61B 1/00; A61B 1/04

(52) U.S. Cl. CPC A61B 1/00188; A61B 1/043

(57) ABSTRACT

The present disclosure provides a triple-wafer dual-band fluorescent zoom adapter for an endoscope, relating to the technical field of biomedicine. The triple-wafer dual-band fluorescent zoom adapter for an endoscope includes a front fixing group, a zooming group, a compensating group and a rear fixing group which are sequentially arranged along an optical axis from an object side to an image side, where an infrared light path and a visible light path are arranged behind the rear fixing group; the zooming group can move along the optical axis to change a focal length; and the compensating group can move along the optical axis to perform correction and focusing of image surface changes accompanying zooming. The present disclosure improves the definition and contrast of imaging.

### CROSS REFERENCE TO RELATED APPLICATION(S)

This patent application claims the benefit and priority of Chinese Patent Application No. 202011392144.8, filed on Dec. 2, 2020, the disclosure of which is incorporated by reference herein in its entirety as part of the present application.

### TECHNICAL FIELD

The present disclosure relates to the technical field of biomedicine, and particularly relates to a triple-wafer dual-band fluorescent zoom adapter for an endoscope.

### BACKGROUND ART

A traditional endoscope adapter is a fixed-focus visible light band lens, and doctors need to replace adapters with different focal lengths (20 mm, 25 mm, 35 mm, and 45 mm) according to actual observation needs of different endoscopes. A near infrared (NIR) indocyanine green (ICG) fluorescence method is widely used in detection of cancer tumors abroad. A detection rate of early cancers can be greatly increased by a fluorescent endoscope, and the detection rate is increased by 4 to 5 times compared with a conventional method. Fluorescence spectrum characteristics can differentiate and diagnose human diseased tissues, cancer tissues, etc.

At present, there is no relevant fluorescent endoscope product in China.

Therefore, there is an urgent need to develop a triple-wafer dual-band fluorescent zoom adapter for an endoscope to solve the above problems in the prior art.

### SUMMARY

An objective of the present disclosure is to provide a triple-wafer dual-band fluorescent zoom adapter for an endoscope to solve the above problems in the prior art and improve the definition and contrast of imaging.

In order to achieve the above objective, the present disclosure provides the following solutions: the present disclosure provides a triple-wafer dual-band fluorescent zoom adapter for an endoscope, including a front fixing group, a zooming group, a compensating group and a rear fixing group which are sequentially arranged along an optical axis from an object side to an image side, wherein an infrared light path and a visible light path are arranged behind the rear fixing group; the zooming group can move along the optical axis to change a focal length; and the compensating group can move along the optical axis to perform correction and focusing of image surface changes accompanying zooming.

Preferably, the front fixing group may include a first lens and a second lens, the first lens may have a negative focal power, and the second lens may have a positive focal power; an object side surface of the first lens may be a convex surface, an image side surface of the first lens may be a concave surface, an object side surface of the second lens may be a convex surface, and an image side surface of the second lens may be a concave surface; the image side surface of the first lens and the object side surface of the second lens may be cemented with each other to form a first cemented lens; and

the first cemented lens may meet vd2−vd1>22, where vd1 and vd2 may respectively represent dispersion coefficients of the first lens and the second lens on a line d.

Preferably, the zooming group may include a third lens and a fourth lens, the third lens may have a positive focal power, and the fourth lens may have a negative focal power; an object side surface of the third lens may be a convex surface, an image side surface of the third lens may be a concave surface, an object side surface of the fourth lens may be a convex surface, and an image side surface of the fourth lens may be a concave surface; the image side surface of the third lens and the object side surface of the fourth lens may be cemented with each other to form a second cemented lens; and

the second cemented lens may meet vd3−vd4>18, where vd3 and vd4 may respectively represent dispersion coefficients of the third lens and the fourth lens on a line d.

Preferably, the compensating group may include a fifth lens and a sixth lens, the fifth lens may have a negative focal power, and the sixth lens may have a negative focal power; an object side surface of the fifth lens may be a concave surface, an image side surface of the fifth lens may be a convex surface, an object side surface of the sixth lens may be a concave surface, and an image side surface of the sixth lens may be a concave surface; the image side surface of the fifth lens and the object side surface of the sixth lens may be cemented with each other to form a third cemented lens; and

the third cemented lens may meet vd6−vd5>19, where vd5 and vd6 may respectively represent dispersion coefficients of the fifth lens and the sixth lens on a line d.

Preferably, a diaphragm may be arranged between the compensating group and the rear fixing group.

Preferably, the rear fixing group may include a seventh lens, an eighth lens, a ninth lens and a tenth lens; the seventh lens may have a positive focal power, an object side surface of the seventh lens may be a convex surface, and an image side surface of the seventh lens may be a convex surface; the eighth lens may have a negative focal power, an object side surface of the eighth lens may be a concave surface, and an image side surface of the eighth lens may be a convex surface; the ninth lens may have a positive focal power, an object side surface of the ninth lens may be a convex surface, and an image side surface of the ninth lens may be a convex surface; the tenth lens may have a negative focal power, an object side surface of the tenth lens may be a concave surface, and an image side surface of the tenth lens may be a convex surface;

the seventh lens and the eighth lens may be cemented to form a fourth cemented lens, and the ninth lens and the tenth lens may be cemented to form a fifth cemented lens;

the fourth cemented lens may meet vd7−vd8>22, where vd7 and vd8 may respectively represent dispersion coefficients of the seventh lens and the eighth lens on a line d; and

the fifth cemented lens may meet vd9−vd10>30, where vd9 and vd10 may respectively represent dispersion coefficients of the ninth lens and the tenth lens on a line d.

Preferably, the infrared light path may include a beam splitter prism, a compensating mirror and an infrared charge coupled device (CCD); the beam splitter prism may include two congruent isosceles right-angled triangular prisms attached to each other, and inclined surfaces of the isosceles right-angled triangular prisms may be coated with films to realize reflection of visible light and transmission of infrared light; and the compensating mirror may adopt flat glass, may be attached to the beam splitter prism, and may be used to compensate for an optical distance of the infrared light path, and the infrared light may enter the infrared CCD after passing through the compensating mirror.

Preferably, the visible light path may include triple wafers, and the triple wafers may be used to realize beam splitting filtration of the visible light from the beam splitter prism to split the visible light into red light, green light and blue light.

Preferably, the triple wafers may include a first prism, a second prism and a third prism which are cemented together, the first prism may be coated with a red light band reflecting film, the third prism may be coated with a green light reflecting film, and the second prism may be coated with a blue light anti-reflection film.

Preferably, the zooming group and the compensating group may be installed on a mobile station.

Compared with the prior art, the present disclosure has the following beneficial technical effects:

In the present disclosure, a four-group structure is adopted to simplify the number of lenses in each group and shorten a focusing distance of the lens; a total length of the lens is reduced to within 80 mm to realize continuous changes in focal length; and furthermore, the definition of imaging is further improved by the triple wafers.

### BRIEF DESCRIPTION OF THE DRAWINGS

To describe the technical solutions in the embodiments of the present disclosure or in the prior art more clearly, the accompanying drawings required for the embodiments are briefly described below. Apparently, the accompanying drawings in the following description show merely some embodiments of the present disclosure, and persons of ordinary skill in the art may still derive other accompanying drawings from these accompanying drawings without creative efforts.

FIG. 1 is a schematic diagram of structure compositions of the present disclosure.

FIG. 2 is an enlarged diagram of a triple-wafer structure of the present disclosure.

FIG. 3 is an optical schematic diagram of the present disclosure at a longest focal length.

FIG. 4 is an optical schematic diagram of the present disclosure at a middle focal length.

FIG. 5 is an optical schematic diagram of the present disclosure at a shortest focal length.

FIG. 6 is a modulation transfer function (MTF) diagram of 0.450-0.680 μm of the present disclosure at the longest focal length.

FIG. 7 is a spot diagram of 0.450-0.680 μm of the present disclosure at the longest focal length.

FIG. 8 is an MTF diagram of 0.810-0.850 μm of the present disclosure at the longest focal length.

FIG. 9 is a spot diagram of 0.810-0.850 μm of the present disclosure at the longest focal length.

FIG. 10 is an MTF diagram of 0.450-0.680 μm of the present disclosure at the middle focal length.

FIG. 11 is a spot diagram of 0.450-0.680 μm of the present disclosure at the middle focal length.

FIG. 12 is an MTF diagram of 0.810-0.850 μm of the present disclosure at the middle focal length.

FIG. 13 is a spot diagram of 0.810-0.850 μm of the present disclosure at the middle focal length.

FIG. 14 is an MTF diagram of 0.450-0.680 μm of the present disclosure at the shortest focal length.

FIG. 15 is a spot diagram of 0.450-0.680 μm of the present disclosure at the shortest focal length.

FIG. 16 is an MTF diagram of 0.810-0.850 μm of the present disclosure at the shortest focal length.

FIG. 17 is a spot diagram of 0.810-0.850 μm of the present disclosure at the shortest focal length.

Brief description of the drawings: L1—first lens, L2—second lens, L3—third lens, L4—fourth lens, L5—fifth lens, L6—sixth lens, L7—seventh lens, L8—eighth lens, L9—ninth lens, L10—tenth lens, L11—beam splitter prism, L12—compensating mirror, L13—infrared CCD, 1—first prism, 2—second prism, 3—third prism, G1—front fixing group, G2—zooming group, G3—compensating group, G4—rear fixing group, A1—infrared light path, A2—visible light path.

### DETAILED DESCRIPTION OF THE EMBODIMENTS

The technical solutions in the embodiments of the present disclosure are clearly and completely described below with reference to the accompanying drawings in the embodiments of the present disclosure. Apparently, the described embodiments are merely a part rather than all of the embodiments of the present disclosure. All other embodiments obtained by persons of ordinary skill in the art on the basis of the embodiments of the present disclosure without creative efforts shall fall within the protection scope of the present disclosure.

To make the above objectives, features and advantages of the present disclosure clearer and more comprehensible, the present disclosure is described in further detail below with reference to the accompanying drawings and specific implementations.

The “a lens has a positive focal power (or a negative focal power)” as described in the present disclosure means that a paraxial focal power of the lens calculated by the Gaussian optical theory is positive (or negative). An “object side surface (or image side surface) of the lens” is defined as a specific range of imaging light passing through a surface of the lens. The convex and concave surfaces of the lens can be judged according to a judging mode of persons of ordinary skill in the art, that is, the concave and convex surface of the lens can be judged by a sign of a radius of curvature (abbreviated as an R value). The R value can be commonly used in optical design software, such as Zemax or Code V. The R value is also commonly used in a lens data sheet of the optical design software. For the object side surface, when the R value is a positive value, it is determined that the object side surface is a convex surface; and when the R value is a negative value, it is determined that the object side surface is a concave surface. On the contrary, for the image side surface, when the R value is a positive value, it is determined that the image side surface is a concave surface; and when the R value is a negative value, it is determined that the image side surface is a convex surface.

### Embodiment 1

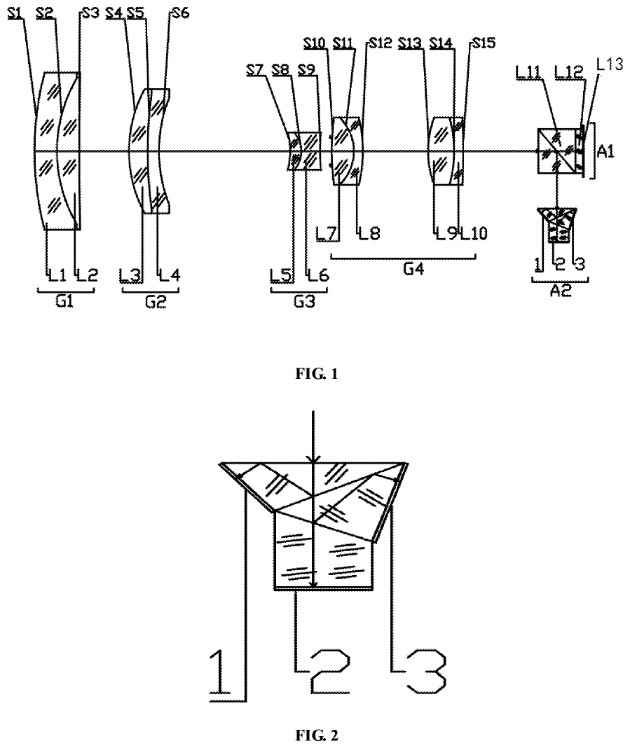

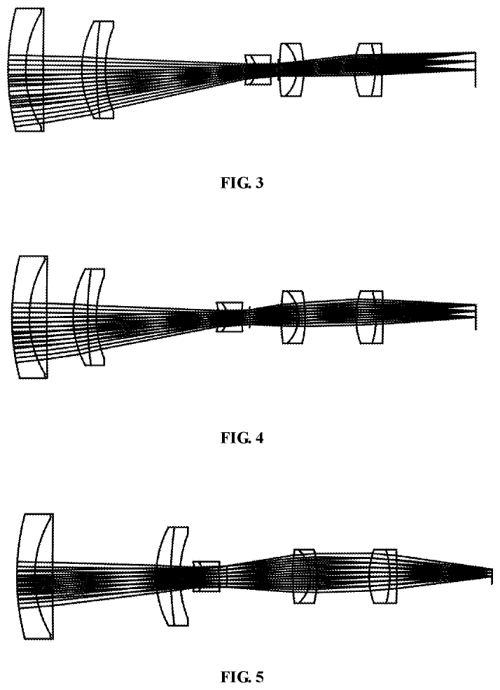

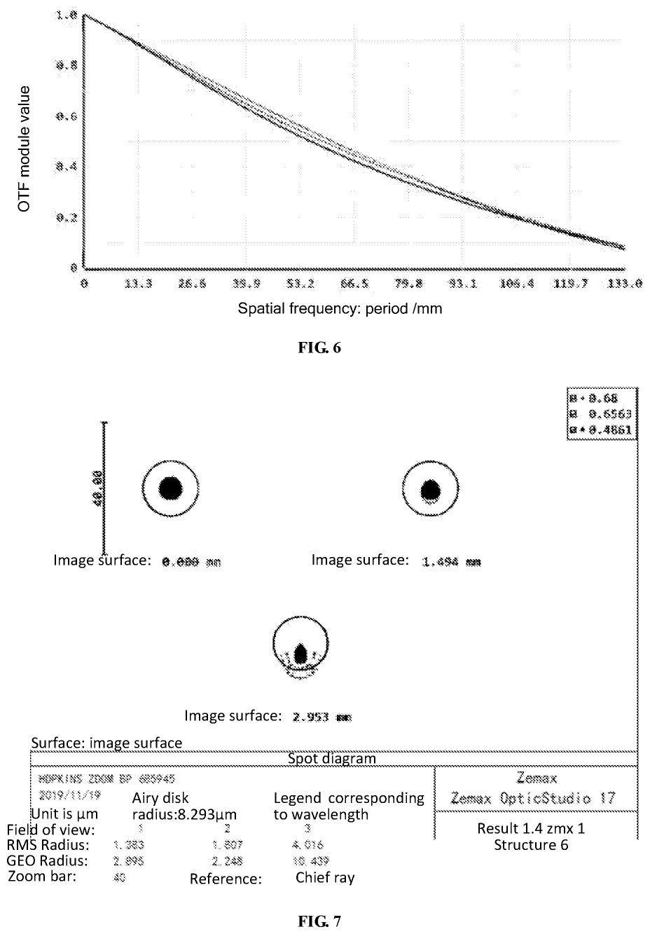

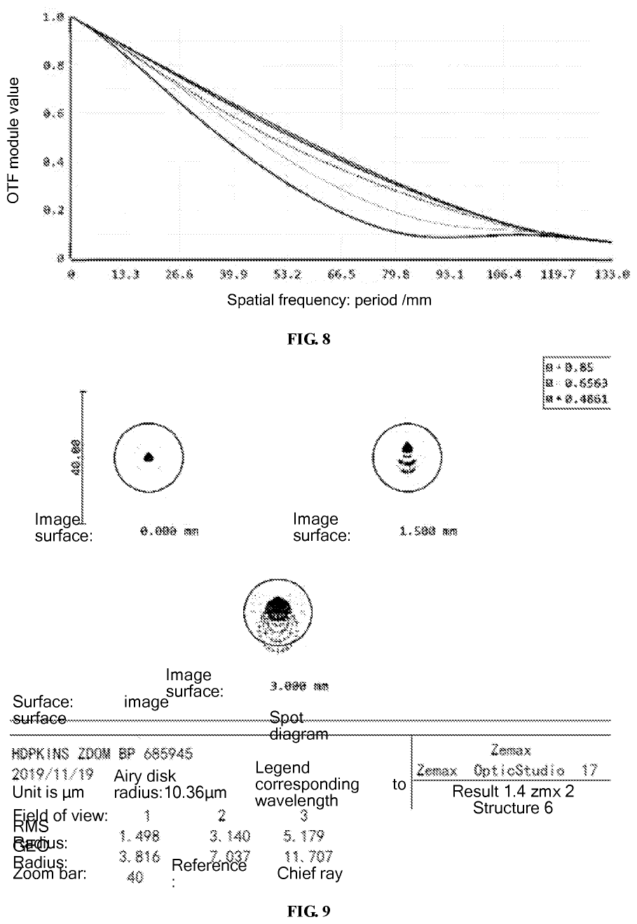

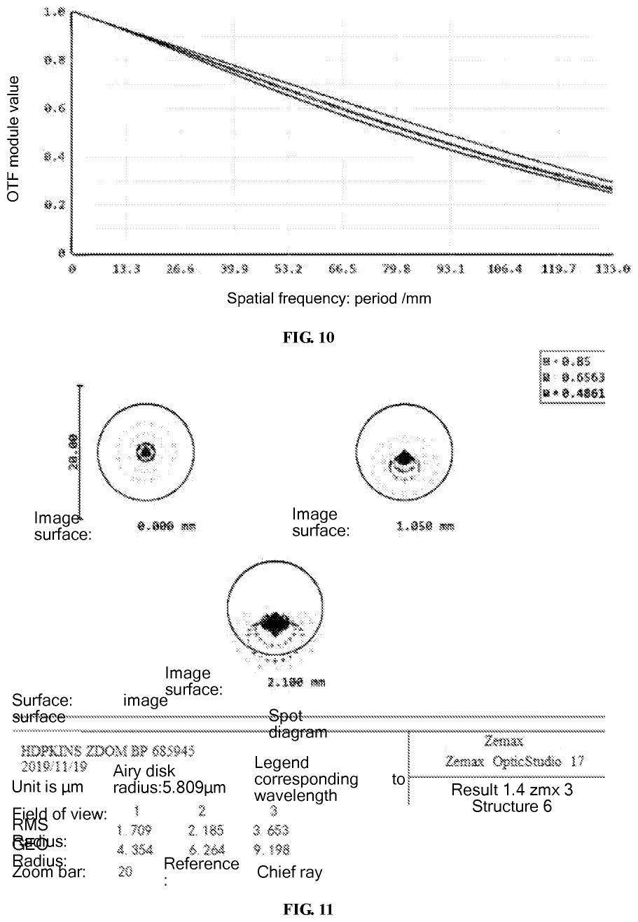

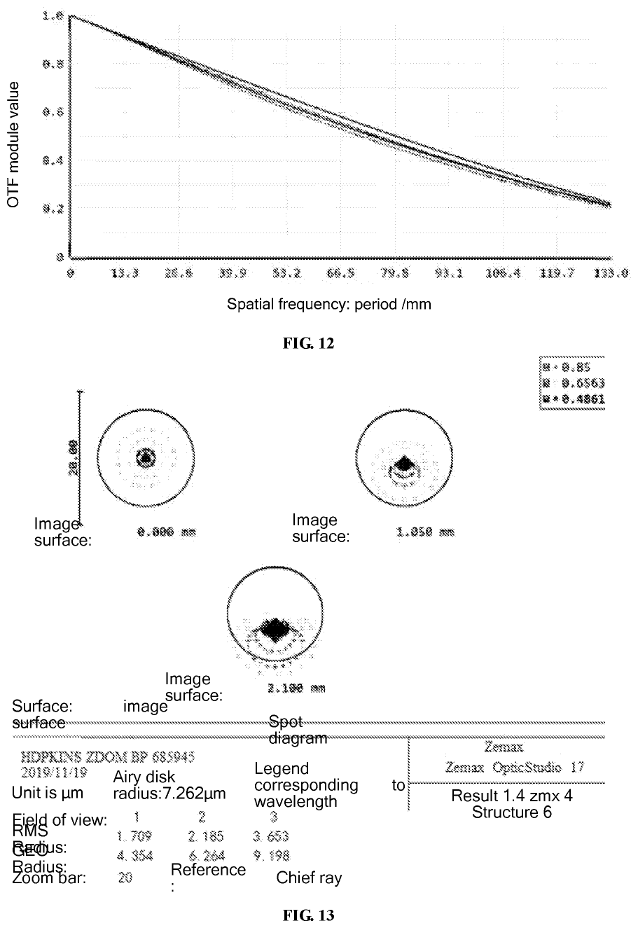

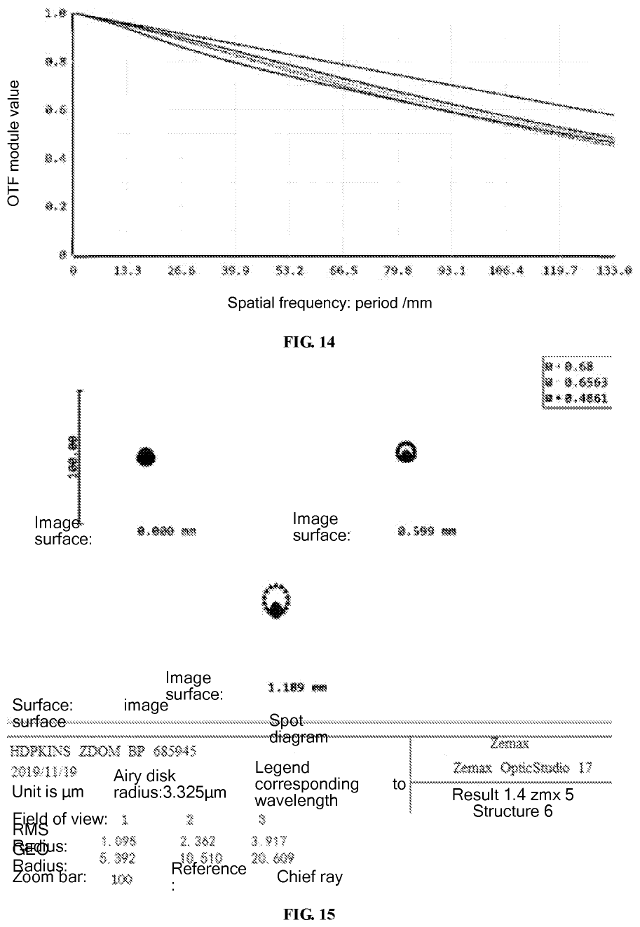

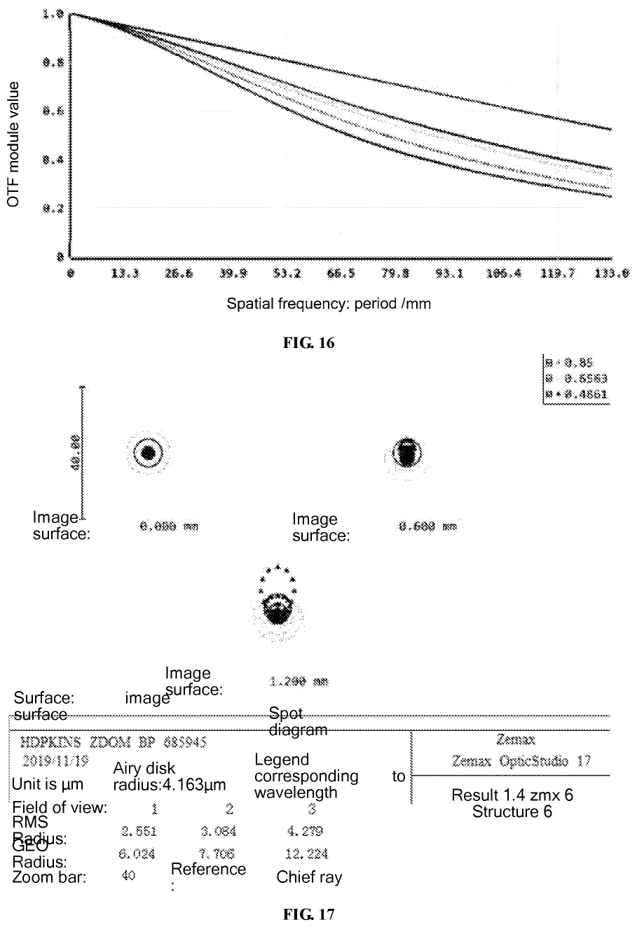

As shown in FIG. 1 to FIG. 17, this embodiment provides a triple-wafer dual-band fluorescent zoom adapter for an endoscope, which is a four-group zoom lens. The change of a focal length of the adapter can be realized by coordinated movement of each lens, dual-band imaging of visible light and infrared light can be realized by a beam splitter prism L11, and the definition of imaging can be improved by a triple-wafer layer-by-layer scanning technology.

In this embodiment, the triple-wafer dual-band fluorescent zoom adapter for an endoscope includes an infrared light path A1 composed of a front fixing group G1, a zooming group G2, a compensating group G3, a diaphragm, a rear fixing group G4, a beam splitter prism L11, a compensating mirror L12 and an infrared CCD which are sequentially arranged along an optical axis from an object side to an image side, and a visible light path A2 composed of triple wafers. In this embodiment, each of a first lens L1 to a tenth lens L10 includes an object side surface which faces the object side and allows imaging light to pass through, and an image side surface which faces the image side and allows the imaging light to pass through.

The front fixing group G1 includes a first lens L1 having a negative focal power and a second lens L2 having a positive focal power, and the image side surface of the first lens L1 and the object side surface of the second lens L2 are cemented to each other to form a first cemented lens.

The object side surface of the first lens L1 is a convex surface, the image side surface of the first lens L1 is a concave surface, a ratio of a focal length of the first lens L1 to an effective aperture of the object side surface is preferably (−3.10, −2.89), and a refractive index of the first lens L1 is preferably greater than 1.6.

The object side surface of the second lens L2 is a convex surface, the image side surface of the second lens L2 is a concave surface, a ratio of a focal length of the second lens L2 to an effective aperture of the object side surface is preferably (1.78, 1.90), and a refractive index of the second lens L2 is preferably greater than 1.5.

The first cemented lens is formed by cementing two cemented lenses having positive and negative focal powers. Through the combination of a refractive index and an Abbe number of a lens material, a chromatic aberration of an optical system is greatly reduced, and a requirement of a consumer-grade lens to focus on chromatic aberration restoration is realized. The first cemented lens further meets vd2−vd1>22, where vd1 and vd2 respectively represent dispersion coefficients of the first lens L1 and the second lens L2 on a line d.

The zooming group G2 includes a third lens L3 having a positive focal power and a fourth lens L4 having a negative focal power, and the image side surface of the third lens L3 and the object side surface of the fourth lens L4 are cemented to each other to form a second cemented lens.

The object side surface of the third lens L3 is a convex surface, the image side surface of the third lens L3 is a concave surface, a ratio of a focal length of the third lens L3 to an effective aperture of the object side surface is preferably (2.58, 2.83), and a refractive index of the third lens L3 is preferably greater than 1.5.

The object side surface of the fourth lens L4 is a convex surface, the image side surface of the fourth lens L4 is a concave surface, a ratio of a focal length of the fourth lens L4 to an effective aperture of the object side surface is preferably (−3.26, −2.98), and a refractive index of the fourth lens L4 is preferably greater than 1.6.

The second cemented lens further meets vd3−vd4>18, where vd3 and vd4 respectively represent dispersion coefficients of the third lens L3 and the fourth lens L4 on the line d.

The compensating group G3 includes a fifth lens L5 having a negative focal power and a sixth lens L6 having a negative focal power, and the image side surface of the fifth lens L5 and the object side surface of the sixth lens L6 are cemented to each other to form a third cemented lens.

The object side surface of the fifth lens L5 is a concave surface, the image side surface of the fifth lens L5 is a convex surface, a ratio of a focal length of the fifth lens L5 to an effective aperture of the object side surface is preferably (−2.00, −1.85), and a refractive index of the fifth lens L5 is preferably greater than 1.6.

The object side surface of the sixth lens L6 is a concave surface, the image side surface of the sixth lens L6 is also a concave surface, a ratio of a focal length of the sixth lens L6 to an effective aperture of the object side surface is preferably (−4.91, −4.58), and a refractive index of the sixth lens L6 is preferably greater than 1.5.

The third cemented lens further meets vd6−vd5>19, where vd5 and vd6 respectively represent dispersion coefficients of the fifth lens L5 and the sixth lens L6 on the line d.

The diaphragm is located between the compensating group G3 and the rear fixing group G4.

The rear fixing group G4 includes a fourth cemented lens and a fifth cemented lens, a seventh lens L7 having a positive focal power and an eighth lens L8 having a negative focal power are cemented to form the fourth cemented lens, and a ninth lens L9 having a positive focal power and a tenth lens L10 having a negative focal power are cemented to form the fifth cemented lens.

The object side surface of the seventh lens L7 is a convex surface, the image side surface of the seventh lens L7 is also a convex surface, a ratio of a focal length of the seventh lens L7 to an effective aperture of the object side surface is preferably (3.40, 4.25), and a refractive index of the seventh lens L7 is preferably greater than 1.5.

The object side surface of the eighth lens L8 is a concave surface, the image side surface of the eighth lens L8 is a convex surface, a ratio of a focal length of the eighth lens L8 to an effective aperture of the object side surface is preferably (−5.57, −5.20), and a refractive index of the eighth lens L8 is preferably greater than 1.6.

The fourth cemented lens further meets vd7−vd8>22, where vd7 and vd8 respectively represent dispersion coefficients of the seventh lens L7 and the eighth lens L8 on the line d.

The object side surface of the ninth lens L9 is a convex surface, the image side surface of the ninth lens L9 is also a convex surface, a ratio of a focal length of the ninth lens L9 to an effective aperture of the object side surface is preferably (2.34, 2.84), and a refractive index of the ninth lens L9 is preferably greater than 1.5.

The object side surface of the tenth lens L10 is a concave surface, the image side surface of the tenth lens L10 is a convex surface, a ratio of a focal length of the tenth lens L10 to an effective aperture of the object side surface is preferably (−5.62, −4.17), and a refractive index of the tenth lens L10 is preferably greater than 1.7.

The fifth cemented lens further meets vd9−vd10>30, where vd9 and vd10 respectively represent dispersion coefficients of the ninth lens L9 and the tenth lens L10 on the line d.

In the infrared light path, the beam splitter prism L11 splits the dual-band light from the tenth lens L10, the transmitted infrared light enters the compensating mirror L12 to reach an infrared CCD L13, and visible light is reflected to enter the triple wafers.

The triple wafers form a beam splitter prism group, and beam splitting filtration of the visible light is realized by three prisms. The visible light reflected by the beam splitter prism L11 is split into red light, blue light and green light which are respectively imaged on red, green and blue CCDs, and the definition of imaging is improved by a layer-by-layer scanning technology.

Specifically, the triple wafers include a first prism 1, a second prism 2 and a third prism 3 which are cemented together. In order to achieve an effect of light splitting, the first prism 1 is coated with a red light band reflecting film allowing other visible light to pass through; the third prism 3 is coated with a green light reflecting film allowing visible light in other bands to pass through; the second prism 2 is coated with a blue light anti-reflection film; and other visible light absorbing films do not allow light in other bands to pass through.

A zoom range of the triple-wafer dual-band fluorescent zoom adapter for an endoscope meets 1.25<f/BFL<3.13, where f represents a focal length, and BFL represents a back focal length at each focal length.

In this embodiment, the zooming group G2 and the compensating group G3 are installed on a mobile station and are driven by the mobile station to move. A specific structure of the mobile station is selected according to work needs. In this embodiment, in a process of changing a focal length, the zooming group G2 changes linearly, and accordingly, the compensating group G3 changes non-linearly. In this embodiment, a back distance of the image side surface of the second lens L2, a distance of the image side surface of the fourth lens L4 and a back distance of a diaphragm surface are three variables during a zooming process.

In this embodiment, in the first lens L1 to the tenth lens L10, two lenses are usually cemented to each other to better control a chromatic aberration, and the triple-wafer dual-band fluorescent zoom adapter for an endoscope only includes the above ten lenses. This embodiment can realize a clear color image, good control on a transfer function, a high resolution, a high definition, a high image sharpness, a uniform image, and strong switching flexibility.

Various numerical data regarding the zoom lens of the embodiment are shown below.

At a longest focal length, EFL=50 mm, FNo=5, and BFL=16 mm.

At a middle focal length, EFL=35 mm, FNo=7, and BFL=16 mm.

At a shortest focal length, EFL=20 mm, FNo=4, and BFL=16 mm.

TABLE 1
Structural Parameters of Lens of This Embodiment

| Surface | | Radius of | | |
|---|---|---|---|---|
| Number | Surface Type | Curvature | Thickness | Material |
| S1 | Spherical Surface | 41.474 | 3.000 | F7 |
| S2 | Spherical Surface | 19.315 | 3.000 | BAK1 |
| S3 | Spherical Surface | 576.385 | 17.435-4.553 | |
| S4 | Spherical Surface | 17.440 | 2.500 | BAK1 |
| S5 | Spherical Surface | 58.367 | 1.500 | BASF1 |
| S6 | Spherical Surface | 19.409 | 2.571-21.077 | |
| S7 | Spherical Surface | −9.695 | 1.500 | BASF1 |
| S8 | Spherical Surface | −3.737 | 2.500 | BAK1 |
| S9 | Spherical Surface | 25.474 | 1.358 | |
| Diaphragm | — | Inf | 11.067-0.198 | |
| S10 | Spherical Surface | 33.891 | 2.965 | BAK1 |
| S11 | Spherical Surface | −6.224 | 1.200 | F7 |
| S12 | Spherical Surface | −18.783 | 8.704 | |
| S13 | Spherical Surface | 12.783 | 3.500 | BK7 |
| S14 | Spherical Surface | −16.334 | 1.200 | SF15 |
| S15 | Spherical Surface | −88.435 | 16.000 | |

TABLE 2
Zoom Parameters of Lens of This Embodiment

| | Focal | | Image | Movement of | Movement of |
|---|---|---|---|---|---|
| | Length | BFL | Height | Zooming | Compensating |
| Type | (mm) | (mm) | (mm) | Group (mm) | Group (mm) |
| Shortest | 20 | 16 | 2.4 | 4.553 | 5.443 |
| Focal | | | | | |
| Length | | | | | |
| Middle | 35 | 16 | 4.2 | 6.628 | 0.198 |
| Focal | | | | | |
| Length | | | | | |
| Longest | 50 | 16 | 6.0 | 17.435 | 11.067 |
| Focal | | | | | |
| Length | | | | | |

TABLE 3
Parameters of Beam Splitter Prism, Compensating
Mirror and Triple Wafers

| Type | Size | Material |
|---|---|---|
| Beam Splitter Prism | Size of Right-angle Side: 10 mm | H-K9L |
| | Angle of Inclination: 45° | |
| | Thickness: 10 mm | |
| Compensating Mirror | Aperture: 10 mm | H-K9L |
| | Thickness: 1.5 mm | |
| Triple Wafers | Incident Light Surface: 10 mm | H-K9L |
| | Aperture of First Surface: 4.4 mm | |
| | Aperture of Second Surface: 5.2 mm | |
| | Aperture of Third Surface: 6.0 mm | |

It should be noted that it is obvious to those skilled in the art that the present disclosure is not limited to the details of the above exemplary embodiments, and that the present disclosure can be implemented in other specific forms without departing from the spirit or basic features of the present disclosure. Therefore, the embodiments should be regarded as exemplary and non-limiting in every respect. The scope of the present disclosure is defined by the appended claims rather than the above description, therefore, all changes falling within the meaning and scope of equivalent elements of the claims should be included in the present disclosure, and any reference numbers in the claims should not be construed as a limitation to the claims involved.

Specific examples are used for illustration of the principles and implementations of the present disclosure. The description of the above embodiments is merely used to help understand the method and its core ideas of the present disclosure. In addition, persons of ordinary skill in the art can make modifications in terms of specific implementations and scope of use according to the ideas of the present disclosure. In conclusion, the content of this specification should not be construed as a limitation to the present disclosure.

What is claimed is:

1. A triple-wafer dual-band fluorescent zoom adapter for an endoscope, comprising a front fixing group, a zooming group, a compensating group and a rear fixing group which are sequentially arranged along an optical axis from an object side to an image side, wherein an infrared light path and a visible light path are arranged behind the rear fixing group; the zooming group can move along the optical axis to change a focal length; and the compensating group can move along the optical axis to perform correction and focusing of image surface changes accompanying zooming.

2. The triple-wafer dual-band fluorescent zoom adapter for an endoscope according to claim 1, wherein the front fixing group comprises a first lens and a second lens, the first lens has a negative focal power, and the second lens has a positive focal power; an object side surface of the first lens is a convex surface, an image side surface of the first lens is a concave surface, an object side surface of the second lens is a convex surface, and an image side surface of the second lens is a concave surface; the image side surface of the first lens and the object side surface of the second lens are cemented with each other to form a first cemented lens; and
the first cemented lens meets vd2−vd1>22, wherein vd1 and vd2 respectively represent dispersion coefficients of the first lens and the second lens on a line d.

3. The triple-wafer dual-band fluorescent zoom adapter for an endoscope according to claim 1, wherein the zooming group comprises a third lens and a fourth lens, the third lens has a positive focal power, and the fourth lens has a negative focal power; an object side surface of the third lens is a convex surface, an image side surface of the third lens is a concave surface, an object side surface of the fourth lens is a convex surface, and an image side surface of the fourth lens is a concave surface; the image side surface of the third lens and the object side surface of the fourth lens are cemented with each other to form a second cemented lens; and
the second cemented lens meets vd3−vd4>18, wherein vd3 and vd4 respectively represent dispersion coefficients of the third lens and the fourth lens on a line d.

4. The triple-wafer dual-band fluorescent zoom adapter for an endoscope according to claim 1, wherein the compensating group comprises a fifth lens and a sixth lens, the fifth lens has a negative focal power, and the sixth lens has a negative focal power; an object side surface of the fifth lens is a concave surface, an image side surface of the fifth lens is a convex surface, an object side surface of the sixth lens is a concave surface, and an image side surface of the sixth lens is a concave surface; the image side surface of the fifth lens and the object side surface of the sixth lens are cemented with each other to form a third cemented lens; and
the third cemented lens meets vd6−vd5>19, wherein vd5 and vd6 respectively represent dispersion coefficients of the fifth lens and the sixth lens on a line d.

5. The triple-wafer dual-band fluorescent zoom adapter for an endoscope according to claim 1, wherein a diaphragm is arranged between the compensating group and the rear fixing group.

6. The triple-wafer dual-band fluorescent zoom adapter for an endoscope according to claim 1, wherein the rear fixing group comprises a seventh lens, an eighth lens, a ninth lens and a tenth lens; the seventh lens has a positive focal power, an object side surface of the seventh lens is a convex surface, and an image side surface of the seventh lens is a convex surface; the eighth lens has a negative focal power, an object side surface of the eighth lens is a concave surface, and an image side surface of the eighth lens is a convex surface; the ninth lens has a positive focal power, an object side surface of the ninth lens is a convex surface, and an image side surface of the ninth lens is a convex surface; the tenth lens has a negative focal power, an object side surface of the tenth lens is a concave surface, and an image side surface of the tenth lens is a convex surface;
the seventh lens and the eighth lens are cemented to form a fourth cemented lens, and the ninth lens and the tenth lens are cemented to form a fifth cemented lens;
the fourth cemented lens meets vd7−vd8>22, wherein vd7 and vd8 respectively represent dispersion coefficients of the seventh lens and the eighth lens on a line d; and
the fifth cemented lens meets vd9−vd10>30, wherein vd9 and vd10 respectively represent dispersion coefficients of the ninth lens and the tenth lens on a line d.

7. The triple-wafer dual-band fluorescent zoom adapter for an endoscope according to claim 1, wherein the infrared light path comprises a beam splitter prism, a compensating mirror and an infrared charge coupled device (CCD); the beam splitter prism comprises two congruent isosceles right-angled triangular prisms attached to each other, and inclined surfaces of the isosceles right-angled triangular prisms are coated with films to realize reflection of visible light and transmission of infrared light; and the compensating mirror adopts flat glass, is attached to the beam splitter prism, and is used to compensate for an optical distance of the infrared light path, and the infrared light enters the infrared CCD after passing through the compensating mirror.

8. The triple-wafer dual-band fluorescent zoom adapter for an endoscope according to claim 7, wherein the visible light path comprises triple wafers, and the triple wafers are used to realize beam splitting filtration of the visible light from the beam splitter prism to split the visible light into red light, green light and blue light.

9. The triple-wafer dual-band fluorescent zoom adapter for an endoscope according to claim 8, wherein the triple wafers comprise a first prism, a second prism and a third prism which are cemented together, the first prism is coated with a red light band reflecting film, the third prism is coated with a green light reflecting film, and the second prism is coated with a blue light anti-reflection film.

10. The triple-wafer dual-band fluorescent zoom adapter for an endoscope according to claim 1, wherein the zooming group and the compensating group are installed on a mobile station.
